(19) United States
(12) Patent Application Publication
(10) Pub. No.: US 2024/0418713 A1
(43) Pub. Date: Dec. 19, 2024

(54) COLLOIDAL GOLD IMMUNOCHROMATOGRAPHIC TEST STRIP FOR DETECTING ENRAMYCIN, PREPARATION METHOD, AND APPLICATION THEREOF

(71) Applicants: ZHEJIANG GONGSHANG UNIVERSITY, Hangzhou City (CN); HANGZHOU RUIQI BIOTECHNOLOGY CO., LTD., Hangzhou City (CN)

(72) Inventors: Min CHEN, Hangzhou City (CN); Hongmei LI, Hangzhou City (CN); Kang WANG, WANG (CN)

(73) Assignees: ZHEJIANG GONGSHANG UNIVERSITY, Hangzhou City (CN); HANGZHOU RUIQI BIOTECHNOLOGY CO., LTD., Hangzhou City (CN)

(21) Appl. No.: 18/652,824

(22) Filed: May 2, 2024

(30) Foreign Application Priority Data
Jun. 14, 2023 (CN) 2023107105018

(51) Int. Cl. G01N 33/543; G01N 33/68

(52) U.S. Cl. CPC G01N 33/54387; G01N 33/68

(57) ABSTRACT

The invention provides a colloidal gold immunochromatographic test strip for detecting enramycin, a preparation method, and an application thereof, which belongs to the field of rapid immunoassay; the test strip comprises a backing plate, a sample pad, a conjugate pad, a nitrocellulose membrane, and an absorbent pad; the conjugate pad was fixed with colloidal gold-labeled enramycin monoclonal antibody; the nitrocellulose membrane is equipped with a T-line coated with enramycin artificial antigens and a C-line coated with goat anti-mouse secondary antibodies. The invention also provides a kit for detecting enramycin, comprising a test strip, a sample treatment solution, and a sample diluent. The invention further adds a test strip shooting background board to the kit, which can be used for rapid qualitative or quantitative detection of enramycin in animal feed and products of animal meat and egg with a detection limit of 10 mg/k

### TECHNICAL FIELD

The invention belongs to the field of rapid immunoassay, especially relates to a colloidal gold immunochromatographic test strip for detecting enramycin, a preparation method, and an application thereof.

### BACKGROUND ART

Enramycin, secreted by Streptomyces fungicidious, is a polypeptide-binding organic compound composed of 17 amino acids and 13 fatty acids. Enramycin has strong antibacterial activity against Gram-positive bacteria such as Clostridium, Streptococcus, and Streptococcus pneumoniae, and can effectively regulate the gastrointestinal flora of various animals, it is mainly used as an antibiotic growth promoter (AGP) in feed for livestock and poultry animals. Although enramycin can be used for feed addition, it is often overused in the pursuit of benefits in livestock and poultry breeding, which will not only damage the organs and tissues of animals but also increase its residue in animal products. Therefore, in order to ensure the safety of animal food, it is urgent to establish a rapid qualitative/quantitative detection method for enramycin in animal food.

At present, the detection technology of enramycin mainly comprises a microbial method and an instrumental analysis method, but both have a series of shortcomings such as long detection time and expensive equipment, which limit the popularization and large-scale application of the detection method. In recent years, colloidal gold immunochromatography technology has been widely used in the field of animal-derived food quality control, food pathogenic microorganism detection, food virus disease diagnosis, food grain mycotoxin detection, and other food residue detection. However, the use of colloidal gold immunochromatography technology for the detection of enramycin in animal feed or animal-derived food has not been reported. Although the traditional colloidal gold immunochromatographic test paper card can achieve rapid qualitative detection, it still needs to use relevant instruments to complete the quantitative analysis of the test paper card. However, the traditional colloidal gold test paper card reader may have problems such as abnormal light source, inconvenient external power supply, and inconvenient carrying in outdoor detection, and in extreme weather, it may cause damage to the instrument due to the lack of waterproof and dustproof measures, thus affecting the accuracy of the analysis results. Therefore, how to simply and quickly use colloidal gold immunochromatography to quantitatively detect enramycin in animal feed or animal-derived food is one of the urgent problems to be solved in this field.

### SUMMARY

In view of this, the purpose of the invention is to provide a colloidal gold immunochromatographic test strip for detecting enramycin, which has strong specificity, and other enramycin structural analogs do not interfere with the detection signal.

The purpose of the invention is also to provide a kit for detecting enramycin, the test results are not required to be read by complex instruments, and the sensitivity is high up to 10 ng/mL.

The purpose of the invention is also to provide a method for detecting enramycin, which can be used for rapid qualitative or quantitative detection of enramycin in animal feed and products of animal meat and eggs, and the detection limit can reach 10 mg/kg.

In order to achieve the above purpose, the invention provides the following technical solution:

The invention provides a colloidal gold immunochromatographic test strip for detecting enramycin, the test strip comprises a backing plate, a sample pad, a conjugate pad, a nitrocellulose membrane, and an absorbent pad, the conjugate pad is fixed with colloidal gold labeled enramycin monoclonal antibodies; the nitrocellulose membrane is equipped with a T-line coated with enramycin artificial antigens and a C-line coated with goat anti-mouse secondary antibodies.

Preferably, a diameter of gold nanoparticles on the conjugate pad is 40-60 nm; a coating concentration of colloidal gold labeled enramycin monoclonal antibodies is 4 μg/mL.

Preferably, the artificial antigen is obtained by coupling enramycin A and BSA protein by carbonyl diimidazole method, a coating concentration of the artificial antigens is 1.0 mg/mL.

The invention also provides a preparation method for the colloidal gold immunochromatographic test strip, overlapping the sample pad, the conjugate pad, the nitrocellulose membrane and the absorbent pad in turn on the backing plate, and the T-line is located on a side near the conjugate pad, soaking and drying the sample pad in a solution containing 0.5-1.5% BSA, 1.5-2.5% sucrose, and 1.5-2.5% S9 in advance.

The invention also provides a kit for detecting enramycin, comprising a sample treatment solution, a sample diluent, and a colloidal gold immunochromatographic test strip mentioned above;
the sample treatment solution is 0.01 M PBS solution containing 50% methanol; the sample diluent is 0.01 M PBS solution containing 1% Tween 20 and 1% S9.

Preferably, the kit comprises a test strip shooting background board; the background board consists of a pure black background, a white positioning frame, a standard black color block, a standard white color block, and a test card frame.

The invention also provides a method for detecting enramycin adopting the above kit, the detection steps comprise: adding the sample treatment solution to a sample to be tested, after oscillation, ultrasonication, and centrifugation, filtering an obtained supernatant to obtain a sample extract; adding the sample diluent to the sample extract, and obtaining a sample solution by mixing, detecting the sample solution by the colloidal gold immunochromatographic test strip.

Preferably, the sample to be tested comprises animal feed and products of animal meat and eggs.

Preferably, a detection result of the colloidal gold immunochromatographic test strip is qualitatively judged by naked eyes; or the test strip is placed on a test strip shooting background board and read quantitatively by a program.

Preferably, a quantitative reading method is as follows: identifying actual gray values of standard black color block, standard white color block, T-line, and C-line by OpenCV algorithm, and combining actual gray values of standard black color block and standard white color block with theoretical gray values to construct a calibration curve, inputting the actual gray values of T-line and C-line the calibration curve to obtain a theoretical gray value of T-line, and then calculating a content of enramycin in the sample.

The beneficial effects of the invention are as follows:

The enramycin colloidal gold immunochromatographic test strip and its kit described in this invention have strong specificity, and other enramycin structural analogs do not interfere with the detection signal. Only when enramycin is present in the sample to be tested, the colloidal gold-labeled antibody will bind by enramycin in the sample and cannot be captured by the probe coated on the detection line.

The enramycin colloidal gold immunochromatographic test strip and its kit described in the invention are simple to use, easy to operate, and do not require special experimental conditions. It can observe the qualitative results with naked eyes or read the quantitative results with the supporting colloidal gold recognition software within 15 min and has a high sensitivity of up to 10 ng/mL. It can be used for large-scale on-site inspection and has a good application prospect in the field of rapid immunization.

The enramycin detection kit described in the invention combines the OpenCV algorithm with the help of intelligent devices (mobile phones, computers, etc.) for rapid quantitative detection of enramycin, which can greatly reduce the data error caused by environmental changes affecting the accuracy of the instrument.

### BRIEF DESCRIPTION OF THE DRAWINGS

FIG. 1 is a side structure diagram of the enramycin colloidal gold immunochromatographic test strip;

FIG. 2 is a coloration principle of the enramycin colloidal gold immunochromatographic test strip;

FIG. 3 is a shooting background board of the enramycin colloidal gold immunochromatographic test strip;

FIG. 4 is a schematic diagram of the signal recognition process of the enramycin colloidal gold immunochromatographic test strip;

FIG. 5 is a sensitivity test result of the enramycin colloidal gold immunochromatography kit;

FIG. 6 is a specific detection result of the enramycin colloidal gold immunochromatography kit;

FIG. 7 is a display interface of the enramycin detection program recognizing the negative result;

FIG. 8 is a display interface of the enramycin detection program recognizing the weak positive result; and

FIG. 9 is a display interface of the enramycin detection program recognizing the positive result.

### DETAILED DESCRIPTION OF THE EMBODIMENTS

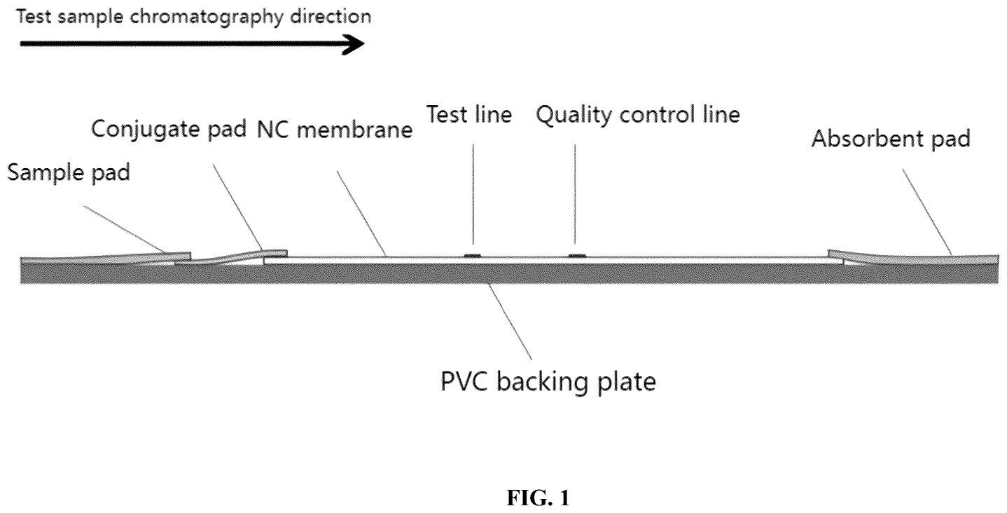

The invention discloses a colloidal gold immunochromatographic test strip for detecting enramycin (the side structure diagram is shown in FIG. 1), the test strip comprises a backing plate, a sample pad, a conjugate pad, a nitrocellulose membrane, and an absorbent pad, the conjugate pad is fixed with colloidal gold labeled enramycin monoclonal antibodies; the nitrocellulose membrane is equipped with a T-line (detection line) coated with enramycin artificial antigens and a C-line (quality control line) coated with goat anti-mouse secondary antibodies.

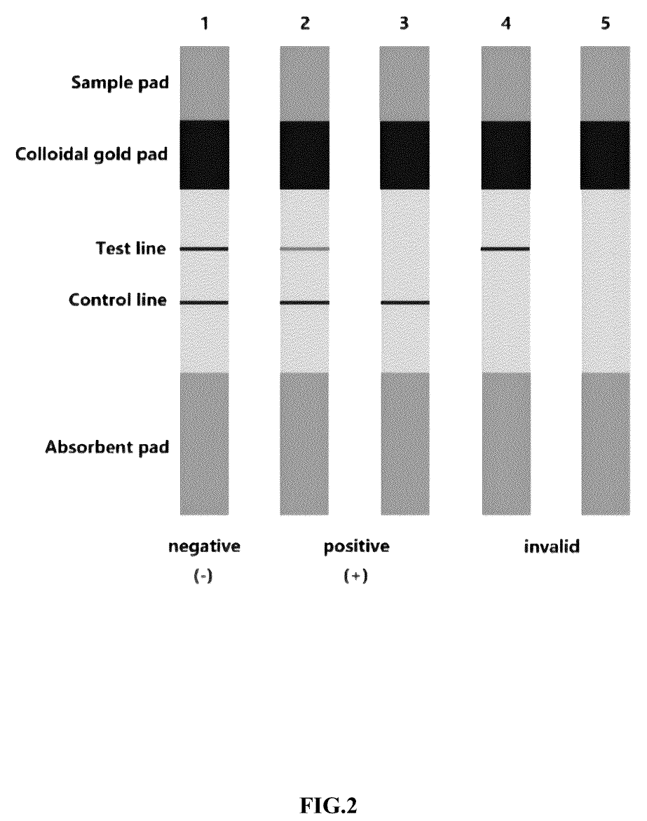

The coloration principle of the test strip described in this invention is shown in FIG. 2. After the sample pad is placed in the sample solution, the sample solution is chromatographed in the direction of the absorbent pad due to the chromatography effect. When the sample solution reaches the conjugate pad, the enramycin A contained in the sample solution is combined with the colloidal gold-labeled enramycin monoclonal antibody to form a complex, which is chromatographed with the sample solution. As the content of enramycin in the sample to be tested increases, the colloidal gold-labeled antibodies on the conjugate pad will first bind to the enramycin in the sample, and the residual antibody will bind to the artificail antigen at the T-line, so the color of the T-line will gradually become lighter and disappear with the increase of the content of enramycin in the sample to be tested. The antigen-antibody complex and the unbinding gold-labeled antibodies will continue to be chromatographed with the sample solution and combined with the secondary antibody on the quality control line, and the C-line will be colored, when the color signal on the T-line is weaker, the content of enramycin in the sample solution is higher.

The test strip of the invention is preferably 2-4 mm wide, and more preferably 3 mm.

The lengths of the absorbent pad, nitrocellulose membrane, conjugate pad, and sample pad are preferably 35-37 mm, 19-21 mm, 8-10 mm, and 17-19 mm respectively, and more preferably 36 mm, 20 mm, 9 mm and 18 mm, the distance between the T-line and the C-line described in the invention is 8-12 mm, preferably 9-10 mm.

The diameter of gold nanoparticles on the conjugate pad is 40-60 nm, preferably 50 nm, and the coating concentration of the colloidal gold-labeled enramycin monoclonal antibody is 4 μg/mL.

As an optional implementation method, the preparation method for the colloidal gold-labeled enramycin monoclonal antibody described in the invention is as follows: the colloidal gold particles are drawn in a beaker, and K₂CO₃ is added under a stirring condition to adjust the pH of the solution to 7.0, the stir condition is continued to carry out, the enramycin monoclonal antibodies are added to make the antibody concentration of the final reaction solution reach 4 μg/mL, and then the solution is fully mixed and marked; BSA is added until the final concentration is 0.5%, and the reaction is carried out for 40 min; the supernatant is discarded twice by centrifugation, and the precipitate is dissolved with the colloidal gold complex solution, diluted to constant volume, and stored at 4° C. for later use.

Preferably, the enramycin monoclonal antibodies are obtained by immunizing BALB/c mice with a hybridoma cell line 2H1F91 (CGMCC No. 15785).

The artificial antigen is obtained by coupling enramycin A and BSA protein by carbonyldiimidazole method, the coating concentration of the artificial antigens is 1.0 mg/mL.

The invention also provides the preparation method for the colloidal gold immunochromatographic test strip, including the following steps: the sample pad, the conjugate pad, the nitrocellulose membrane, and the absorbent pad are overlapped in turn on the backing plate, and the T-line is located on the side near the conjugate pad. As an optional implementation method, the invention binds the nitrocellulose membrane to the middle of the PVC backing plate, pastes the conjugate pad at the T-line end, pastes the sample pad at the other end of the conjugate pad, and pastes the absorbent pad at the C-line end, the pasted overlapped part of the invention is 1-3 mm, preferably 2 mm.

The sample pad of the invention is soaked and dried in a solution containing 0.5-1.5% BSA, 1.5-2.5% sucrose, and 1.5-2.5% S9 in advance, and stored at 4° C., preferably, the solution contains 1% BSA, 2% sucrose and 2% S9, the soaking time is 1 hour, and preferably, the drying method is drying in 37 degrees Celsius. S9 of the invention is the surfactant S9 with a trade name of pluracare1307; preferably, the sample pad is glass fiber.

The conjugate pad of the invention is pre-sprayed with colloidal gold-labeled enramycin monoclonal antibody for standby. The spraying amount is 3 μL/cm; the spraying method is a film-spraying gold-spraying machine. Preferably, the conjugate pad of the invention is glass fiber.

The nitrocellulose membrane T-line of the invention is pre-sprayed with artificial antigen, and the spraying amount is 0.7 μL/cm. The spraying method is spraying by a film-spraying gold-spraying all-in-one machine; the nitrocellulose membrane C-line of the invention is pre-sprayed with goat anti-mouse secondary antibodies, and the spraying amount is 0.7 μL/cm, the spraying method is spraying by a film-spraying gold-spraying machine. The invention does not limit the source of goat anti-mouse secondary antibodies.

The invention provides a kit for detecting enramycin, comprising a sample treatment solution, a sample diluent, and a colloidal gold immunochromatographic test strip; the sample treatment solution of the invention is 0.01 M PBS solution containing 50% methanol; the sample diluent is 0.01 M PBS solution containing 1% Tween 20 and 1% S9.

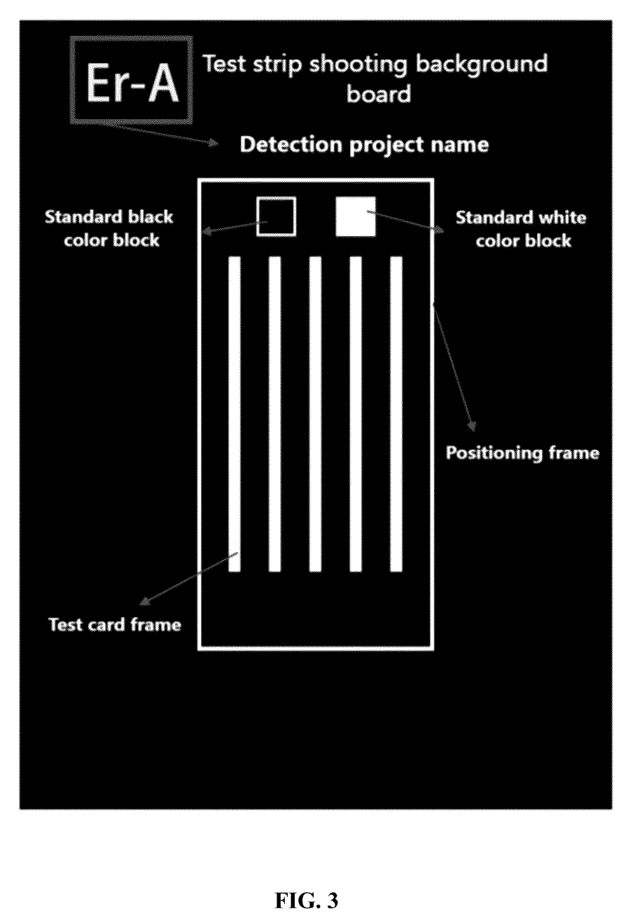

The kit also comprises a test strip shooting background board (see FIG. 3); the background board consists of a pure black background, a white positioning frame, a standard black color block, a standard white color block, and a test card frame. As an optional implementation method, the white positioning frame of the invention is 6 cm×12 cm, the standard black color block is 1 cm×1 cm, the standard white color block is 1 cm×1 cm, and the test card frame is 0.3 cm×8 cm. The actual area of the card frame of the invention can be changed according to the shape of the test paper card actually used.

The invention also provides a method for detecting enramycin, the method comprises: the sample treatment solution is added to the sample to be tested, and after oscillation, ultrasonication, and centrifugation, the obtained supernatant is filtered to obtain a sample extract; the sample diluent is added to the sample extract, and a sample solution is obtained by mixing, the sample solution is detected by the colloidal gold immunochromatographic test strip. Preferably, the sample treatment solution is added to the sample to be tested, and supernatant I and precipitate I are obtained after oscillation, ultrasonication, and centrifugation, the sample treatment solution is added again to precipitate I, and supernatant II is obtained by oscillation, ultrasonication, and centrifugation, and supernatant I and supernatant II are combined. Preferably, the supernatant is filtered by a filter membrane to obtain a sample extract.

The sample to be tested in this invention comprises animal feed and products of animal meat and egg, the mass volume ratio of the sample to be tested to the sample treatment solution is 1:1.5-3 g/mL, which is preferably 1:2 g/mL.

Preferably, the oscillation of the invention is oscillated by a vortex oscillator, the oscillation time is 10-20 min, preferably 12-15 min. Preferably, the ultrasonic treatment described in the invention is room temperature ultrasound, and the ultrasonic time is 8-12 min, preferably 10 min. Preferably, the centrifugation described in the invention is centrifuging for 8-12 min at a centrifugal force of 8000 g and 4° C. The invention centrifuges to obtain supernatant I and precipitate I, and supernatant I is reserved; the sample treatment solution is added again to precipitation I, and the above treatment is repeated to obtain supernatant II, the supernatant obtained by merging the two times is filtered by a 450 nm filter membrane to obtain the sample extract.

The sample extract of the invention is added with 3-5 times the volume of the sample diluent, and the sample solution is obtained by mixing the sample diluent, preferably, the additional amount of the sample diluent is 4 times the volume of the sample extraction liquid; preferably, the mixing method is blowing slowly with a pipette for 8 to 10 times.

The sample solution obtained by the invention is detected by the colloidal gold immunochromatographic test strip, the detection method can be selected as inserting one end of the sample pad of the test strip into the sample solution for 2-5 min or adding the sample droplet to the sample pad of the test strip, the quantitative results are read by naked eyes or qualitative and quantitative results are read by program.

The detection results are obtained after chromatography for 8-15 min, and preferably, the chromatography time is 10-12 min.

The detection results of the invention can be qualitatively judged by naked eyes; the criteria are as follows: when the color of T-line is significantly deeper than that of C-line or as deep as that of C-line, it is negative, and when the color of T-line is weaker than that of C-line, it is positive.

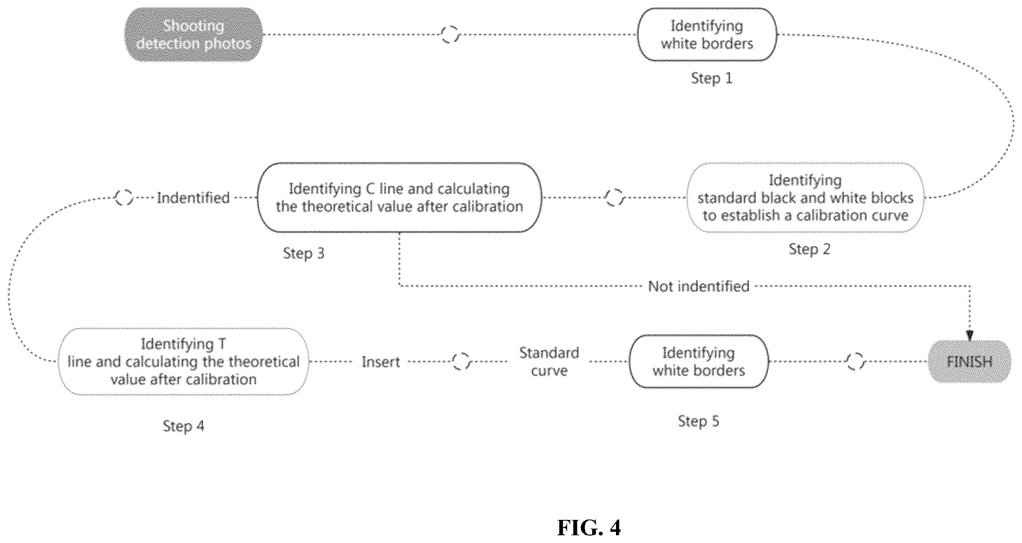

The detection results of the invention can be quantitatively read; the reading method is placing the test strip on the background board of the test strip and read quantitatively by the program. In this invention, the test strip is placed on the background board of the test strip, and the photos are taken from the top with smartphones (it should be noted that there should not be too strong light and shadow contrast in the box marked by the background board during shooting, otherwise it will affect the determination of the results of the experiment). The OpenCV algorithm is used to identify the actual gray value of the standard black color block, the standard white color block, the T-line and the C-line, and the actual gray value and theoretical gray value of the standard black color block and the standard white color block (black: 0; white: 255). The calibration curve is constructed with the theoretical gray values of standard black and white blocks as abscissa and the actual gray values of standard black and white blocks as ordinate. The actual gray values of T-line and C-line are brought into the calibration curve to obtain the calibration gray value, the content of enramycin in the sample is calculated according to the calibration gray value of T-line, the schematic diagram of the processing flow is shown in FIG. 4.

The determination criteria of the quantitative reading of the invention are as follows: (1) The theoretical gray value of the C-line is only used for quality control, when the theoretical gray value of the C-line is less than 40, the determination result is invalid. (2) When the C-line is greater than or equal to 40, the detection is effective, and the T-line results are determined, when the detection is effective, the theoretical gray value of the T-line identified in the program is greater than or equal to 90, it is judged to be negative; when the theoretical gray value of the T-line identified in the program is greater than or equal to 60 and less than 90, it is judged to be weak positive; when the theoretical gray value of the T-line identified in the program is less than 60, it is judged to be positive.

The following will combine the embodiments in the invention to describe the technical solution clearly and completely in the invention. Obviously, the described embodiments are only a part of the embodiments of the invention, not all of the embodiments. Based on the embodiments in this invention, all other embodiments obtained by ordinary technicians in this field without making creative labor belong to the scope of protection of this invention.

The methods used in the following embodiments are all conventional methods if there is no special explanation.

The materials, reagents, etc. used in the following embodiments can be obtained from commercial channels without special instructions.

### Embodiment 1

A colloidal gold immunochromatographic test strip for detecting enramycin: composing of a PVC backing plate, a sample pad, a conjugate pad, a nitrocellulose membrane, and an absorbent pad, the width of the test strip is 3 mm; the lengths of absorbent pad, nitrocellulose membrane, conjugate pad and sample pad are 36 mm, 20 mm, 9 mm and 18 mm, respectively.

### (1) Preparation of Monoclonal Antibody Against Enramycin:

a. Female BALB/c mice aged 8-10 weeks are selected and injected intraperitoneally with 0.5 mL of sterilized liquid paraffin, and then standby for one week.
b. Mice are inoculated intraperitoneally with 0.5 mL of hybridoma cell line 2H1F91(CGMCC No. 15785) at a concentration of 10⁶-10⁷ cells/mL in the logarithmic growth phase.
c. About 7 days later, the abdominal cavity is punctured with a syringe to collect ascites, the cells are centrifuged at 12,000 r/min for 15 min to remove cell debris and large protein aggregates, and the supernatant is collected, the supernatant is first crudely purified by saturated octanoic acid-ammonium sulfate precipitation method, and then purified by Protein GAgarose Resin 4FF protein G agarose rapid purification resin according to the instructions to obtain enramycin monoclonal antibodies.

### (2) Preparation of the Colloidal Gold-Labeled Enramycin Monoclonal Antibodies:

5 mL colloidal gold particles are taken into a beaker and placed on a magnetic stirrer for slow stirring, a certain amount of 0.2 M K₂CO₃ is added to adjust the pH of the solution to 7.0, and the solution is continuously stirred for 10 min. After that, the anti-Enramycin monoclonal antibodies are added to make the final reaction solution antibody concentration reach 4 μg/mL, and marked for 40 min after fully mixing. 10% BSA is added dropwise to the above mixture solution until the final concentration of BSA is set to 0.5%, and then the reaction is performed for 40 min, the solution is transferred to a clean centrifuge tube and centrifuged at 8000 rpm for 40 min; the supernatant is discarded, the rest solution is resuspended with 5 mLPBS buffer and centrifuged at 8000 rpm for 30 min, and then the supernatant is discarded, the precipitate is dissolved with colloidal gold complex solution and diluted to 2 ml, and stored at 4° C. for later use.

### (3) Preparation of the Conjugate Pad:

The conjugate pad is glass fiber, and the colloidal gold-labeled enramycin monoclonal antibodies are sprayed on the conjugate pad by using an all-in-one machine with a spray amount of 3 μL/cm.

### (4) Preparation of the Nitrocellulose Membrane:

a. Preparation of the artificial antigen: 7.20 mg Er-A, 30 mg N,N′-carbonyldiimidazole, and 22 mg DMAP are reacted overnight at room temperature in 2.5 mL N,N-dimethylformamide. Subsequently, the reaction solution is added dropwise to a PBS (0.1 mol/L, pH8.0) solution containing 20 mg BSA under stirring, the mixture is stirred at 4° C. for 24 h and centrifuged at 2500 g to remove the precipitate, the supernatant is dialyzed at 4° C. in 0.01 mol/L PBS (pH7.4) solution for 72 h, and the dialysate is replaced twice a day to obtain enramycin, one-component complete antigen solution (artificial antigen) is stored at −20° C. for later use.
b. T-line and C-line are set on the nitrocellulose membrane, with a distance of 10 mm; the artificial antigen (1 mg/mL) is sprayed at the T-line, and the coating amount is 0.7 μL/cm by using the film-spraying and gold-spraying instrument, the goat anti-mouse secondary antibody is sprayed (0.4 mg/mL concentration, purchased from Symmerfei) at the C-line, the film-spraying gold-spraying all-in-one machine is used with a spraying amount 0.7 μL/cm.

### (5) Sample Pad Preparation:

The sample pad is glass fiber, the sample pad is soaked in a solution containing 1% BSA, 2% sucrose, and 2% S9 for 1 hour, and is taken out and dried until the water content is below 10%, and stored at 4° C.

### (6) Test Strip Assembly:

The nitrocellulose film is bonded to the middle of the PVC backing plate, the conjugate pad is pasted at the end of the T-line, the sample pad is pasted at the other end of the conjugate pad, and the absorbent pad (water absorption paper) is pasted at the end of the C-line, the overlapping part of the paste is 2 mm.

### Embodiment 2

A kit for detecting enramycin, including a sample treatment solution, sample diluent, and colloidal gold immunochromatographic test strip prepared by Embodiment 1;
the sample treatment solution is 0.01 MPBS solution containing 50% methanol, the sample diluent is 0.01 M PBS solution containing 1% Tween 20 and 1% S9.

### Embodiment 3

A kit for detecting enramycin, comprising sample treatment solution, sample diluent, colloidal gold immunochromatographic test strip prepared by Embodiment 1, and test strip shooting background board;
the sample treatment solution is 0.01 M PBS solution containing 50% methanol; the sample diluent is 0.01 M PBS solution containing 1% Tween 20 and 1% S9.

The test strip shooting background board consists of a pure black background, a white positioning frame (6 cm×12 cm), a standard black color block (1 cm×1 cm), a standard white color block (1 cm×1 cm), and five test card frames (0.3 cm×8 cm).

### Embodiment 4

A method for detecting enramycin (using the kit described in Embodiment 2):

5 g chicken feed to be tested is weighed and placed in a centrifuge tube, and 10 mL of sample treatment solution is added. First, the vortex oscillator is shaken for 15 min, and then ultrasound is performed at room temperature for 10 min, then it is centrifuged at 8000 g and 4° C. for 10 min, and the supernatant is collected for later use, the precipitate after centrifugation is treated twice in the same way. After it is completed, the two supernatants are mixed and the supernatant is filtered with a 450 nm filter membrane to obtain a sample extract.

When detecting, 15 μL sample extract is placed in a micropore, 60 μL sample diluent is added, and the sample solution is obtained after ten times of slow blowing with a pipette, the sample droplets are added to the sample pad of the test strip, and the sample is taken out after 15 min of chromatography, and the results are judged by naked eyes, when the color of T-line is significantly deeper than that of C-line or as deep as that of C-line, it is negative, and when the color of T-line is weaker than that of C-line, it is positive.

### Embodiment 5

The sensitivity of the kit described in Embodiment 2 is verified:

10 parts of 5 g pig feeds are weighed respectively, it is confirmed that the pig feeds weighed above contain no enramycin, those pig feeds are placed in different centrifuge tubes, and enramycin A is added at 0, 0.5, 1, 5, 10, 15, 20, 40, 60, and 80 mg/kg.

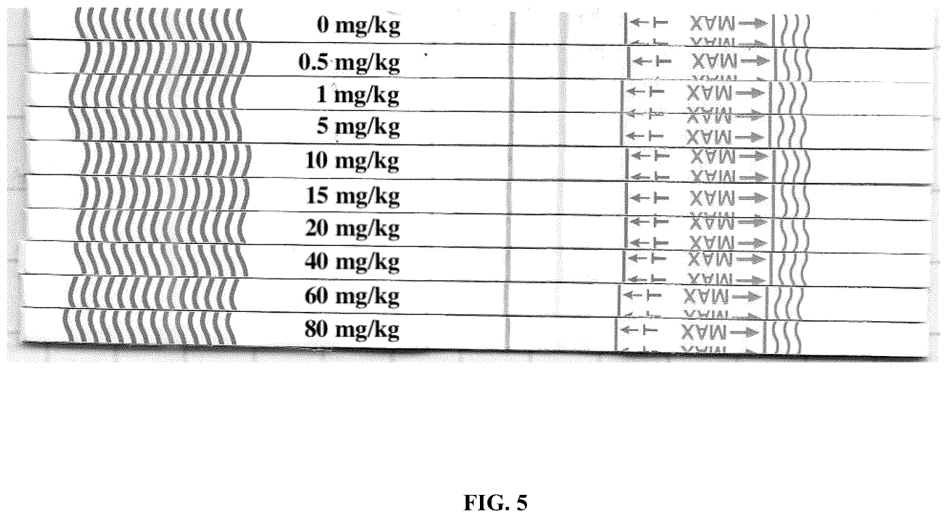

10 mL sample treatment solution is added to each centrifuge tube; first, the vortex oscillator is shaken for 15 min, and then ultrasound is performed at room temperature for 10 min, then it is centrifuged at 8000 g and 4° C. for 10 min, and the supernatant is collected for later use, the sediment after centrifugation is treated in the same way, when it is completed, the two supernatants are mixed and filtered with a 450 nm filter membrane to obtain the sample extract, 15 μL sample extract is placed in micropores, and 60 μL sample diluent is added, the sample solution is obtained after ten times of slow blowing with a pipette, and then the content of enramycin in different sample solutions is detected by high-performance liquid chromatography. At the same time, different sample solutions are detected by the enramycin colloidal gold test card, the results are shown in FIG. 5 (FIG. 5 is the test result of pig feed containing 0, 0.5, 1, 5, 10, 15, 20, 40, 60, 80 mg/kg of enramycin A from left to right).

FIG. 5 shows that when the additional amount of enramycin Ain the feed is 10 mg/kg, the detection line is significantly weakened and the quality control line is normal. At the same time, with the increase of the amount of enramycin A added in the feed, the detection line is completely disappeared, the results show that the detection limit of the kit for enramycin A is 10 mg/kg.

### Embodiment 6

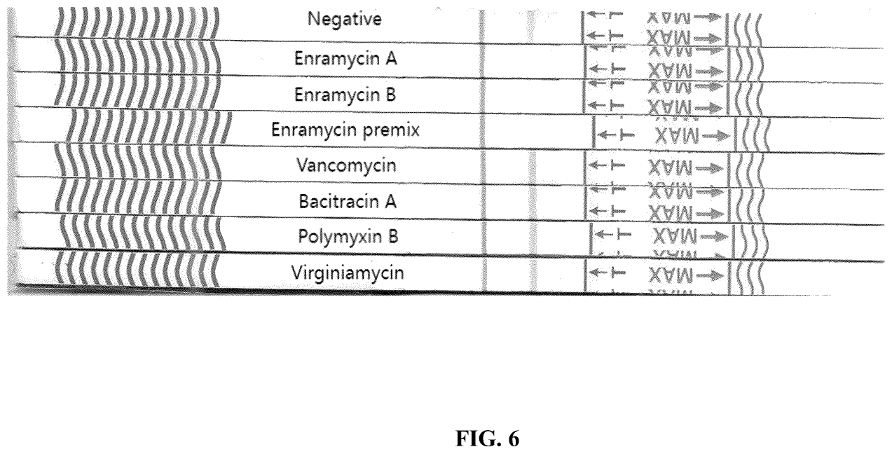

The specificity of the kit described in Embodiment 2 is verified:
enramycin A, enramycin B, enramycin AB mixture, vancomycin, bacitracin A, polymyxin B, and virginiamycin are tested at a concentration of 50 μg/mL for kit speciticity. 15 μL prepared antibiotic solution is taken into the micropores, and 60 μL sample diluent is added and slowly blown ten times. At the same time, the sample pad of the enramycin colloidal gold test strip is inserted into the sample solution, and the coloration principle result is read after 15 min. The results are shown in FIG. 6, the results from left to right are negative control, enramycin A, enramycin B, enramycin premix, vancomycin, bacitracin A, polymyxin B, and virginiamycin respectively.

FIG. 6 shows that the structures of enramycin A and enramycin B are similar, the detection results of enramycin B and enramycin premix are consistent with those of enramycin A, and the detection results of other polypeptide antibiotics are consistent with those of negative control, the enramycin kit prepared by the invention has high specificity and is suitable for the detection of enramycin.

### Embodiment 7

A method for detecting enramycin (using the kit described in Embodiment 3):

The test strips completed by the reaction are placed on the shooting background board in turn, and photographed with a smartphone under a fixed light source to obtain the pictures to be processed.

The establishment of the standard curve needs to be tested with the pre-configured 0, 5, 10, 20, 40, 60, 80, 100, 200, 300 ng/mL of enramycin A standard, after the test is completed, the T-line value is read by the software, the standard curve is established, and the T-line value of the test sample is input into the calculated content.

The image is imported into the signal processing program (a program written in Python language). The program automatically identifies the white box at the outermost edge of the shooting background board for positioning. According to the standard color block on the shooting background board and the green channel of the picture after the color area of the test strip is extracted, the actual gray value of the standard black and white block in the frame is automatically identified by the OpenCV algorithm, and establish a calibration curve is established with its standard gray value (black: 0; white: 255): the theoretical gray value X₁ (0) of the black block, the theoretical gray value X₂ (255) of the white block, the actual gray value Y₁ of the black block and the actual gray value Y₂ of the white block are used to draw a calibration curve, and the calibration curve can be obtained by connecting (X₁, Y₁) and (X₂, Y₂). After obtaining the calibration curve, the algorithm automatically identifies the T-line and C-line of the detection area, and obtains the corresponding actual gray value. First, whether the C-line is valid or not is judged, and if it is invalid, directly output the result of ‘invalid detection’ is output; if it is valid, the actual gray value of the T-line is substituted into the calibration curve to convert the calibration gray value, and then substituted into the established standard curve to obtain the content of enramycin in the sample.

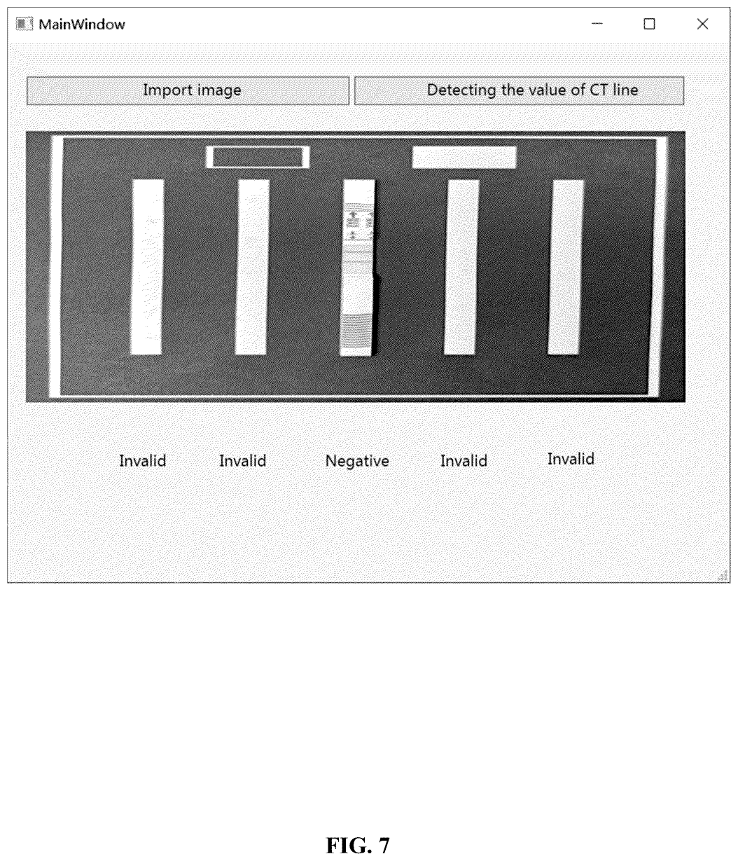

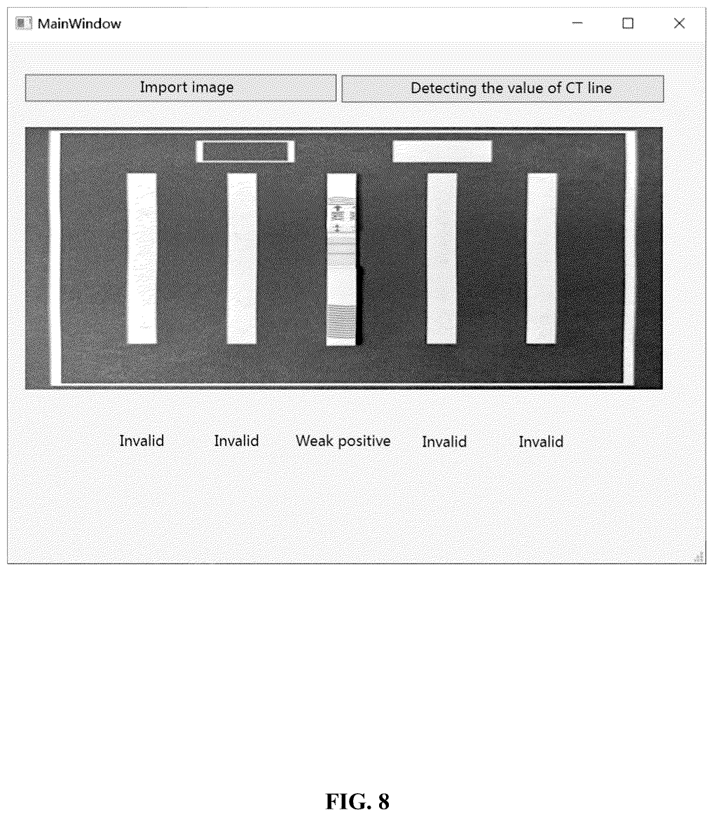

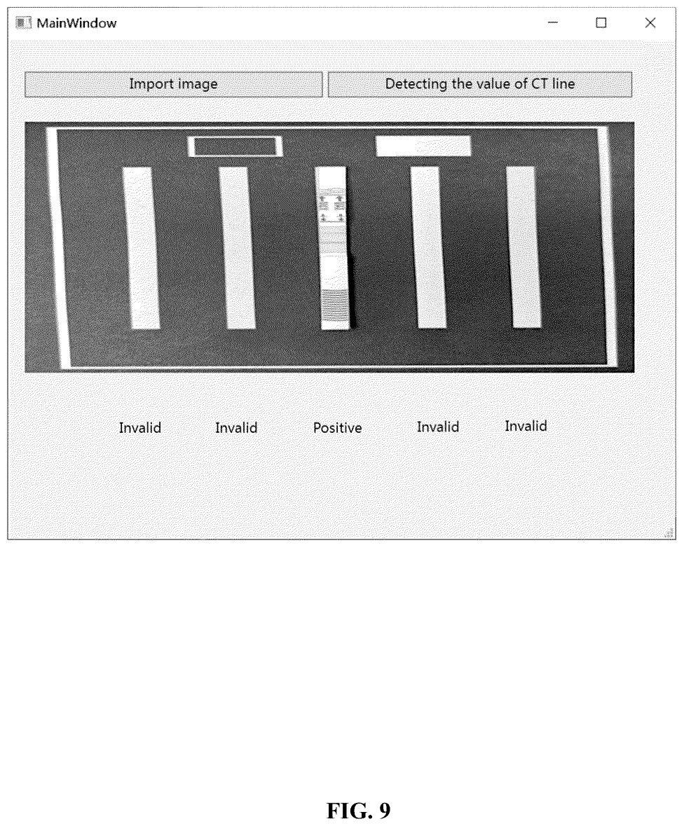

The results are shown in FIGS. 7, 8 and 9. When the photographs are input into the program, the calibration curve y=1.8531x-191.1638 is established by identifying the actual gray value of the standard black and white block. After that, the actual gray values of the identified T and C are brought into the curve to obtain the calibrated T and C values, and the result determination stage is entered: when the C value is greater than or equal to 40, the T value is identified, and when the theoretical gray value of the T-line is greater than or equal to 90, it is judged to be negative; when the theoretical gray value of the T-line is greater than or equal to 60 and less than 90, it is judged to be weak positive; when the theoretical gray value of the T-line less than 60, it is judged to be positive.

According to the results, when the content of enramycin A in the micropore is less than 10 ng/mL, the output is negative; when the content of enramycin A in micropores is greater than or equal to 10 ng/mL and less than 200 ng/mL, the output is weakly positive, when the content of enramycin A in the micropore is greater than or equal to 200 ng/mL, the output is positive, which has a more sensitive detection limit than the method determined by the naked eye.

### Embodiment 8

To verify the stability of the data processing program of the Enramycin colloidal gold test card (using the method described in Embodiment 7):

Program stability tests under different mobile phone models: three types of mobile phones, iPhone 13, Huawei Glory 10, and Xiaomi 12S, are selected to shoot the same experimental results in the above three different lighting environments, and the results are imported into the program for result determination. The results are shown in Table 1:

TABLE 1
Test results of program stability under different mobile phones

| | | | Phone type | | | |
|---|---|---|---|---|---|---|
| | | | iPhone 13 | Huawei Glory 10 | Xiaomi 12S | Accuracy |
| Sample | 0 | ng/mL | Negative | Negative | Negative | 100% |
| concentration | 10 | ng/mL | Weak positive | Weak positive | Weak positive | 100% |
| | 50 | ng/mL | Weak positive | Weak positive | Weak positive | 100% |
| | 200 | ng/mL | Positive | Positive | Positive | 100% |
| | 300 | ng/mL | Positive | Positive | Positive | 100% |

The above content contains only the preferred embodiments of the invention. It should be pointed out that for ordinary technicians in this technical field, some improvements and embellishments can be made without breaking away from the principle of the invention, and these improvements and embellishments should also be regarded within the protection scope of the invention.

What is claimed is:

1. A colloidal gold immunochromatographic test strip for detecting enramycin, the test strip comprises a backing plate, a sample pad, a conjugate pad, a nitrocellulose membrane and an absorbent pad, the conjugate pad is fixed with colloidal gold labeled enramycin monoclonal antibodies; the nitrocellulose membrane is equipped with a T-line coated with enramycin artificial antigens and a C-line coated with goat anti-mouse secondary antibodies.

2. The colloidal gold immunochromatographic test strip according to claim 1, wherein a diameter of gold nanoparticles on the conjugate pad is 40-60 nm; a coating concentration of colloidal gold labeled enramycin monoclonal antibodies is 4 μg/mL.

3. The colloidal gold immunochromatographic test strip according to claim 1, wherein the artificial antigen is obtained by coupling enramycin A and BSA protein by carbonyl diimidazole method, a coating concentration of the artificial antigens is 1.0 mg/mL.

4. A preparation method for the colloidal gold immunochromatographic test strip according to claim 1, overlapping the sample pad, the conjugate pad, the nitrocellulose membrane, and the absorbent pad in turn on the backing plate, and the T-line is located on a side near the conjugate pad, soaking and drying the sample pad in a solution containing 0.5-1.5% BSA, 1.5-2.5% sucrose, and 1.5-2.5% S9 in advance.

5. A kit for detecting enramycin, comprising a sample treatment solution, a sample diluent, and the colloidal gold immunochromatographic test strip according to claim 1;
the sample treatment solution is 0.01 M PBS solution containing 50% methanol; the sample diluent is 0.01 M PBS solution containing 1% Tween 20 and 1% S9.

6. The kit according to claim 5, wherein it comprises a test strip shooting background board; the background board consists of a pure black background, a white positioning frame, a standard black color block, a standard white color block, and a test card frame.

7. A method for detecting enramycin adopting the kit according to claim 5, wherein detection steps comprise: adding the sample treatment solution to a sample to be tested, after oscillation, ultrasonication, and centrifugation, filtering an obtained supernatant to obtain a sample extract; adding the sample diluent to the sample extract, and obtaining a sample solution by mixing, detecting the sample solution by a colloidal gold immunochromatographic test strip.

8. The method according to claim 7, wherein the sample to be tested comprises animal feed and products of animal meat and egg.

9. The method according to claim 7, wherein a detection result of the colloidal gold immunochromatographic test strip is qualitatively judged by naked eyes; or the test strip is placed on a test strip shooting background board and read quantitatively by a program.

10. The method according to claim 9, wherein a quantitative reading method is as follows: identifying actual gray values of standard black color block, standard white color block, T-line, and C-line by OpenCV algorithm, and combining actual gray values of standard black color block and standard white color block with theoretical gray values to construct a calibration curve, inputting the actual gray values of T-line and C-line the calibration curve to obtain a theoretical gray value of T-line, and then calculating a content of enramycin in a sample.
